(19) United States
(12) Patent Application Publication
(10) Pub. No.: US 2024/0081894 A1
(43) Pub. Date: Mar. 14, 2024

(54) TREATMENT TOOL

(71) Applicant: OLYMPUS MEDICAL SYSTEMS CORP., Tokyo (JP)

(72) Inventors: Motomu NAGATA, Tokyo (JP); Ojiro KITAMURA, Tokyo (JP); Satoshi SHIMABUKURO, Saitama-shi (JP)

(21) Appl. No.: 18/461,200

(22) Filed: Sep. 5, 2023

Related U.S. Application Data
(60) Provisional application No. 63/375,135, filed on Sep. 9, 2022.

(51) Int. Cl. A61B 18/14

(52) U.S. Cl. CPC A61B 18/1447; A61B 2017/320093

(57) ABSTRACT

A treatment tool includes: a first gripper; a second gripper configured to pivot relative to the first gripper to grip a living tissue between the second gripper and the first gripper; a support being provided with a pivoting shaft configured to engage with the second gripper and serve as a pivoting center of the second gripper; and a driver provided with a driving shaft configured to engage with the second gripper, the driver being configured to move forward and backward to pivot the second gripper.

### RELATED APPLICATION DATA

This application is based on and claims priority under 35 U.S.C. § 119 to U.S. Provisional Application No. 63/375,135, filed on Sep. 9, 2022, the entire contents of which are incorporated herein by reference.

### FIELD OF DISCLOSURE

The present disclosure relates to a treatment tool.

### BACKGROUND

An example of a treatment tool that treats a treatment target by applying ultrasound energy as treatment energy to a treatment target site (hereinafter, referred to as a treatment target) of living tissues is described for example, JP 11-192238 A.

The treatment tool for treating a treatment target in this manner employs, for example, the following configuration.

More specifically, the treatment tool includes a first gripper and a second gripper that grips a treatment target between the second gripper and the first gripper. Here, the second gripper is configured to be pivotable about a pivoting shaft provided to a support relative to the first gripper. Furthermore, the second gripper grips the treatment target between the second gripper and the first gripper by pivoting about the pivoting shaft. Furthermore, the second gripper engages with a driver by a driving shaft, and pivots about the pivoting shaft in response to forward and backward movement of the driver. Furthermore, in a state where the treatment target is gripped between the first and second grippers, the treatment tool treats the treatment target by applying ultrasound vibration to the treatment target from a treatment portion that is a site on the distal end side of the first gripper.

FIGS. 14A and 14B and FIGS. 15A and 15B are views for describing a conventional problem. In FIGS. 14A and 14B and FIGS. 15A and 15B, reference numeral “100” denotes the above-described treatment tool. Reference numeral “101” denotes the above-described first gripper. Reference numeral “102” denotes the above-described second gripper. Reference numeral “103” denotes the above-described driver. Reference numeral “104” denotes the above-described support. Reference sign “Pi1A” denotes the above-described pivoting shaft. Reference sign “Pi2A” denotes the above-described driving shaft. The reference sign “LT” denotes a treatment target.

However, the above-described treatment tool 100 has the following problems.

When the treatment target LT is gripped between the first and second grippers 101 and 102 (FIG. 14A and FIG. 15A), and then the second gripper 102 is further pivoted in a direction to approach the first gripper 101, the first gripper 101 warps (FIG. 14B and FIG. 15B). Here, in a case where the treatment target LT is thin and soft living tissues, the gripping pressure on the distal end side (the left sides in FIGS. 14A and 14B and FIGS. 15A and 15B) of the treatment target LT is higher than that of other sites as illustrated in FIG. 14B). On the other hand, in a case where the treatment target LT is thick and hard living tissues, a gripping pressure on the proximal end side (the right sides in FIGS. 14A and 14B and FIGS. 15A and 15B) of the treatment target LT is higher than other sites as illustrated in FIG. 15B).

That is, there is a problem that the gripping pressure distribution at a time when the treatment target LT is gripped varies.

### SUMMARY

In some embodiments, a treatment tool includes: a first gripper; a second gripper configured to pivot relative to the first gripper to grip a living tissue between the second gripper and the first gripper; a support that is provided with a pivoting shaft configured to engage with the second gripper and serve as a pivoting center of the second gripper; and a driver that is provided with a driving shaft configured to engage with the second gripper, the driver being configured to move forward and backward to pivot the second gripper. The second gripper includes a first elongated hole that is an elongated hole extending along a first direction, in which the driving shaft is disposed, and that engages with the driving shaft; and a second elongate hole that is an elongated hole extending along a second direction, in which the pivoting shaft is disposed, and that engages the pivoting shaft, as the driver moves forward and backward, the driving shaft is configured to move along the first direction in the first elongated hole while being guided to the first elongated hole, and as the driving shaft moves along the first direction in the first elongated hole, the second gripper is configured to move along the second direction relative to the pivoting shaft while being guided to the second elongated hole.

In some embodiments, a treatment tool includes: a first gripper; a second gripper including a pivoting shaft, the second gripper configured to pivot about the pivoting shaft relative to the first gripper to grip a living tissue between the second gripper and the first gripper; a support configured to engage with the pivoting shaft; and a driver including a driving shaft configured to engage with the second gripper, the driver being configured to move forward and backward to pivot the second gripper.

The second gripper includes a first elongated hole being an elongated hole extending along a first direction, in which the driving shaft is disposed, the first elongated hole engaging the driving shaft, the support includes a second elongate hole being an elongated hole extending along a second direction, in which the pivoting shaft is disposed, the second elongate hole engaging with the pivoting shaft, as the driver moves forward and backward, the driving shaft is configured to move along the first direction in the first elongated hole while being guided to the first elongated hole, and as the driving shaft moves along the first direction in the first elongated hole, the second gripper is configured to move along the second direction while the pivoting shaft is guided to the second elongated hole.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a view illustrating a treatment system according to an embodiment;

FIG. 2 is a view for describing a configuration of a distal end portion of a treatment tool;

FIG. 3 is a view for describing a gripping operation of a grip portion;

FIG. 4 is a view for describing the gripping operation of the grip portion;

FIG. 5 is a view for describing the gripping operation of the grip portion;

FIG. 6 is a view for describing the gripping operation of the grip portion;

FIG. 7 is a view for describing the gripping operation of the grip portion;

FIG. 8 is a view illustrating modified example 1 of the embodiment;

FIG. 9 is a view illustrating modified example 1 of the embodiment;

FIG. 10 is a view for describing modified example 2 of the embodiment;

FIGS. 11A, 11B and 11C are views for describing modified example 2 of the embodiment;

FIGS. 12A, 12B, and 12C are views for describing modified example 3 of the embodiment;

FIG. 13 is a view for describing modified example 4 of the embodiment;

FIGS. 14A and 14B are views for describing a conventional problem; and

FIGS. 15A and 15B are views for describing the conventional problem.

### DETAILED DESCRIPTION

Hereinafter, a mode for carrying out the disclosure (hereinafter, an embodiment) will be described with reference to the drawings. Note that the disclosure is not limited by the embodiment described below. Furthermore, in the description of the drawings, the same portions will be assigned the same reference numerals.

Schematic Configuration of Treatment System

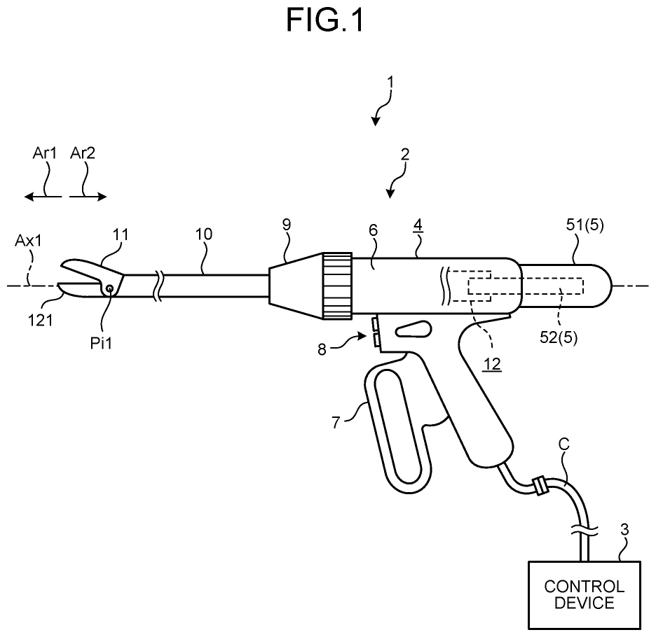

FIG. 1 is a view illustrating a treatment system 1 according to the embodiment.

The treatment system 1 treats a treatment target by applying treatment energy to a treatment target site (hereinafter, referred to as a treatment target) of living tissues. The treatment energy in the present embodiment is ultrasound energy and high frequency energy. Furthermore, treatment that can be executed by the treatment system 1 according to the present embodiment is treatment such as coagulation (sealing) of a treatment target or incision of a treatment target. Furthermore, coagulation and incision may be performed simultaneously. As illustrated in FIG. 1, this treatment system 1 includes a treatment tool 2 and a control device 3.

Configuration of Treatment Tool

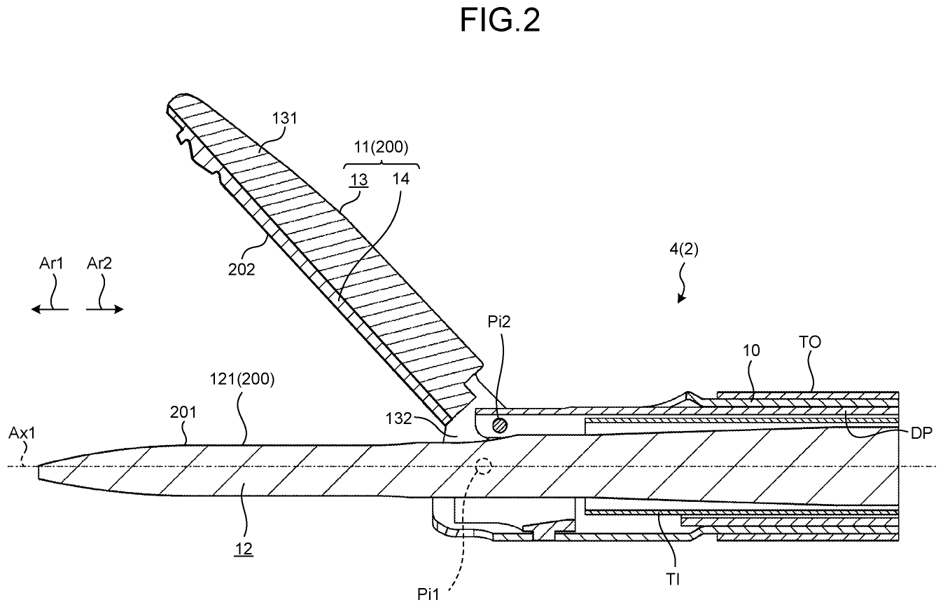

Hereinafter, one side along a center axis Ax1 (FIG. 1) of a sheath 10 is referred to as a distal end side Art, and another side is referred to as a proximal end side Ar2. Furthermore, a “width direction” described below is a direction perpendicular to the center axis Ax1 and an opening/closing direction of a jaw 11 relative to a treatment portion 121, and means a direction perpendicular to the paper faces of FIGS. 1 and 2.

FIG. 2 is a view illustrating a configuration of a distal end portion of the treatment tool 2. More specifically, FIG. 2 is a cross-sectional view illustrating that the distal end portion of the treatment tool 2 is cut along a plane in a state where the jaw 11 and a vibration transmitter 12 are included in the plane including the center axis Ax1 of the sheath 10.

The treatment tool 2 is a treatment tool that treats a treatment target by applying ultrasound energy and high frequency energy to the treatment target. As illustrated in FIG. 1, this treatment tool 2 includes a handpiece 4 and an ultrasound transducer unit 5.

As illustrated in FIGS. 1 and 2, the handpiece 4 includes a fixed handle 6 (FIG. 1), an operation handle 7 (FIG. 1), a switch 8 (FIG. 1), a rotary knob 9 (FIG. 1), the sheath 10, the jaw 11, and the vibration transmitter 12.

The fixed handle 6 is a portion that supports the entire treatment tool 2 and is gripped by an operator (user) such as a surgeon.

The operation handle 7 is movably attached to the fixed handle 6, and accepts an opening/closing operation by the operator such as the surgeon.

The switch 8 is provided in a state where the switch 8 is exposed to the outside of the fixed handle 6, and accepts a treatment operation by the operator such as the surgeon.

The rotary knob 9 has a cylindrical shape that is coaxial with the center axis Ax1, and is provided on the distal end side Ar1 of the fixed handle 6. Furthermore, the rotary knob 9 accepts a rotation operation by the operator such as the surgeon. According to the rotation operation, the rotary knob 9 pivots about the center axis Ax1 relative to the fixed handle 6. Furthermore, rotation of the rotary knob 9 pivots the sheath 10, the jaw 11, and the vibration transmitter 12 about the center axis Ax1.

The sheath 10 has a tubular shape, and corresponds to a support. In the present embodiment, the sheath 10 is a cylindrical pipe made of a conductive material such as metal.

In this sheath 10, a pivoting shaft Pi1 (FIGS. 1 and 2) that is formed as a columnar pin extending in a direction perpendicular to the paper faces of FIGS. 1 and 2, engages with the jaw 11, and pivotally supports the jaw 11 is fixed to an end portion on the distal end side Art.

Furthermore, the outer peripheral surface of the sheath 10 is covered with an outer tube TO having electrical insulation (FIG. 2). Furthermore, a tubular driver DP (FIG. 2) that moves forward and backward along a longitudinal direction of the sheath 10 in response to the opening/closing operation of the operation handle 7 by the operator such as the surgeon is inserted into the sheath 10. A driving shaft Pi2 (FIG. 2) that is formed as a columnar pin extending in a direction perpendicular to the paper faces of FIGS. 1 and 2 and engages with the jaw 11 is fixed to an end portion on the distal end side Ar1 of this driver DP. In the present embodiment, the driving shaft Pi2 is disposed on an upper side (a side on which an arm main body 131 is disposed relative to the treatment portion 121) in FIG. 2 relative to the pivoting shaft Pi1. Furthermore, the inner peripheral surface of the driver DP is covered with an inner tube TI having electrical insulation (FIG. 2).

The jaw 11 corresponds to a second gripper. In the present embodiment, part of the jaw 11 is made of a conductive material. As illustrated in FIG. 2, this jaw 11 includes an arm 13 and a pad 14.

The arm 13 corresponds to a gripper main body. In the present embodiment, the arm 13 is made of a conductive material. As illustrated in FIG. 2, this arm 13 is a member obtained by integrally forming the arm main body 131 and a pair of bearing portions 132.

The arm main body 131 is formed of an elongated plate body, and one plate surface is disposed at a posture facing the vibration transmitter 12.

The pair of bearing portions 132 are provided at each end portion on the proximal end side Ar2 of the arm main body 131, and is formed of plate bodies facing each other in the width direction of the arm main body 131. Note that FIG. 2 illustrates only the one bearing portion 132 of the pair of bearing portions 132. The pair of bearing portions 132 employ the same configuration, and therefore only the configuration of the one bearing portion 132 will be described below.

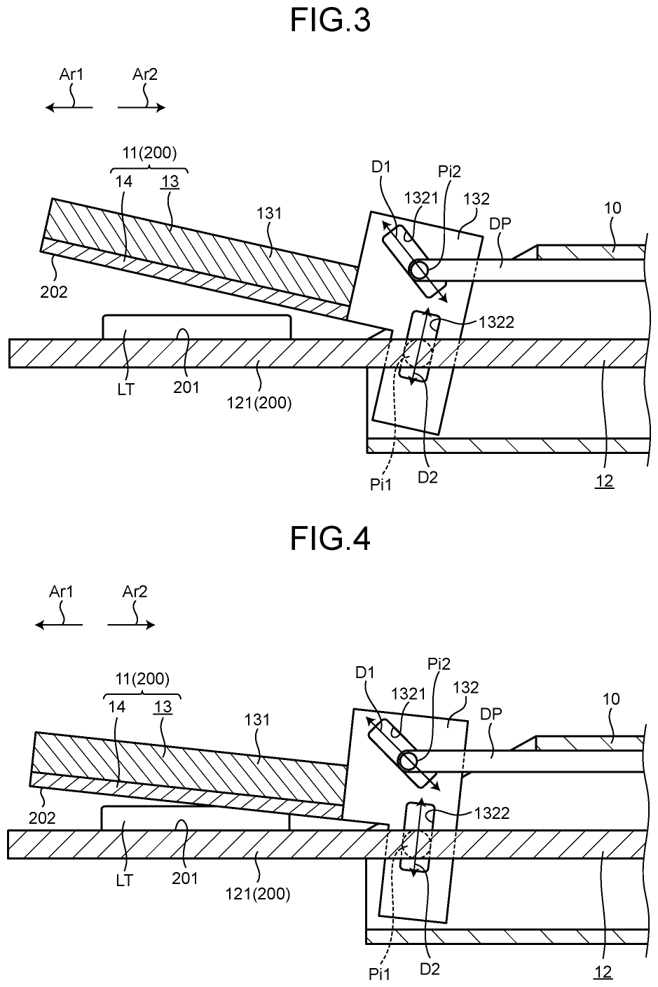

The bearing portion 132 is provided with a second elongated hole 1322 (see FIG. 3) that penetrates the top and the bottom, in which the pivoting shaft Pi1 is disposed (inserted), and that engages with the pivoting shaft Pi1. That is, the arm 13 is connected to the sheath 10 by the pivoting shaft Pi1. This second elongated hole 1322 is an elongated hole that extends linearly along a second direction D2 (see FIG. 3).

Furthermore, the bearing portion 132 is provided with a first elongated hole 1321 (see FIG. 3) that penetrates the top and the bottom, in which the driving shaft Pi2 is disposed (inserted), and that engages with the driving shaft Pi2. That is, the arm 13 is connected to the driver DP by the driving shaft Pi2. This first elongated hole 1321 is an elongated hole that linearly extends along a first direction D1 (see FIG. 3) different from the second direction D2. Also, the first elongated hole 1321 is inclined relative to the second elongated hole 1322.

Furthermore, the arm 13 pivots about the pivoting shaft Pi1 relative to the sheath 10 in conjunction with forward and backward movement of the driver DP in response to the opening/closing operation of the operation handle 7 by the operator such as the surgeon. As a result, the jaw 11 is opened from and closed toward the treatment portion 121 that is an end portion on a distal end side of the vibration transmitter 12, and can grip the treatment target between the jaw 11 and the treatment portion 121. In the present embodiment, the jaw 11 pivots about the pivoting shaft Pi1 in a direction to approach the treatment portion 121 in conjunction with movement of the driver DP to the distal end side Art. That is, the jaw 11 is closed toward the treatment portion 121. Furthermore, the jaw 11 pivots about the pivoting shaft Pi1 in a direction to move away from the treatment portion 121 in conjunction with movement of the driver DP to the proximal end side Ar2. That is, the jaw 11 is opened from the treatment portion 121. As described above, the treatment tool 2 is formed as a so-called push-close type in the present embodiment.

The pad 14 is made of a resin material having electrical insulation and biocompatibility such as PolyTetraFluoroEthylene (PTFE), and has a cuboid shape that extends along the longitudinal direction of the arm 13. Furthermore, as illustrated in FIG. 2, the pad 14 is fixed to the face on the treatment portion 121 side of the arm main body 131, and comes into contact with the treatment portion 121 when the jaw 11 is closed toward the treatment portion 121. This pad 14 has electrical insulation, and therefore has a function of preventing the arm 13 and the vibration transmitter 12 from short-circuiting. Furthermore, the pad 14 has a function of preventing the treatment portion 121 performing ultrasound vibration from colliding against the arm 13 and being damaged when incision of the treatment target by the ultrasound vibration is completed.

Note that, as a method for fixing the pad 14 to the arm main body 131, for example, a fixing method for making a claw portion protrude from the face on the treatment portion 121 side of the arm main body 131 and engaging the pad 14 with the claw portion to mechanically fix, insert molding, or the like can be exemplified.

The vibration transmitter 12 is made of a conductive material, and has an elongated shape that extends along the center axis Ax1. Furthermore, as illustrated in FIG. 2, the vibration transmitter 12 is inserted into the driver DP in a state where the treatment portion 121 protrudes outside. At this time, as illustrated in FIG. 1, the end portion on the proximal end side Ar2 of the vibration transmitter 12 is mechanically connected to an ultrasound transducer 52 that constitutes the ultrasound transducer unit 5. Furthermore, the vibration transmitter 12 transmits the ultrasound vibration generated by the ultrasound transducer unit 5 from the end portion on the proximal end side Ar2 to the treatment portion 121. In the present embodiment, the ultrasound vibration is longitudinal vibration that vibrates in a direction along the center axis Ax1.

Hereinafter, for convenience of description, the jaw 11 and the treatment portion 121 will be referred to as a grip portion 200 (FIG. 2). Furthermore, the flat face on the jaw 11 side of the treatment portion 121 is a face that grips living tissues between the treatment portion 121 and the jaw 11, and corresponds to a first gripping surface 201. Furthermore, the flat face on the treatment portion 121 side of the jaw 11 is a face that grips the living tissues between the jaw 11 and the treatment portion 121, and corresponds to a second gripping surface 202.

Note that details of a gripping operation of the grip portion 200 will be described in “Gripping Operation of Grip Portion” described later.

As illustrated in FIG. 1, the ultrasound transducer unit 5 includes a Transducer (TD) case 51 and the ultrasound transducer 52.

The TD case 51 supports the ultrasound transducer 52, and is detachably connected to the fixed handle 6.

The ultrasound transducer 52 generates ultrasound vibration under control of the control device 3. In the present embodiment, the ultrasound transducer 52 is formed as a Bolted Langevin Transducer (BLT).

Configuration of Control Device

The control device 3 integrally controls the operation of the treatment tool 2 via an electric cable C (FIG. 1).

More specifically, the control device 3 detects a treatment operation of the switch 8 by the operator such as the surgeon via the electric cable C. Furthermore, when detecting the treatment operation, the control device 3 applies treatment energy to the treatment target gripped between the jaw 11 and the treatment portion 121 via the electric cable C. That is, the control device 3 treats the treatment target.

When, for example, applying ultrasound energy to a treatment target, the control device 3 supplies driving power to the ultrasound transducer 52 via the electric cable C. As a result, the ultrasound transducer 52 generates longitudinal vibration (ultrasound vibration) that vibrates in a direction along the center axis Ax1. Furthermore, the longitudinal vibration vibrates the treatment portion 121 with a desired amplitude. Furthermore, the treatment portion 121 applies the ultrasound vibration to the treatment target gripped between the jaw 11 and the treatment portion 121. In other words, the treatment portion 121 applies the ultrasound energy to the treatment target.

Furthermore, when, for example, applying high frequency energy to the treatment target, the control device 3 supplies high frequency power between the arm 13 and the vibration transmitter 12 via the electric cable C, the sheath 10, and the like. Furthermore, when the high frequency power is supplied between the arm 13 and the vibration transmitter 12, a high frequency current flows to the treatment target gripped between the jaw 11 and the treatment portion 121. In other words, the high frequency energy is applied to the treatment target.

Gripping Operation of Grip Portion

Next, the gripping operation of the grip portion 200 described above will be described.

FIGS. 3 to 7 are views for describing the gripping operation of the grip portion 200. More specifically, FIGS. 3 to 7 sequentially illustrate a process in which the jaw 11 is closed toward the treatment portion 121 in response to the opening/closing operation of the operation handle 7 by the operator such as the surgeon.

When the driver DP moves to the distal end side Ar1 in response to the opening/closing operation from a state in FIG. 3 illustrating the state where the operator such as the surgeon does not perform the opening/closing operation on the operation handle 7, the jaw 11 is closed toward the treatment portion 121. As a result, the jaw 11 starts gripping a treatment target LT between the jaw 11 and the treatment portion 121 as illustrated in FIG. 4.

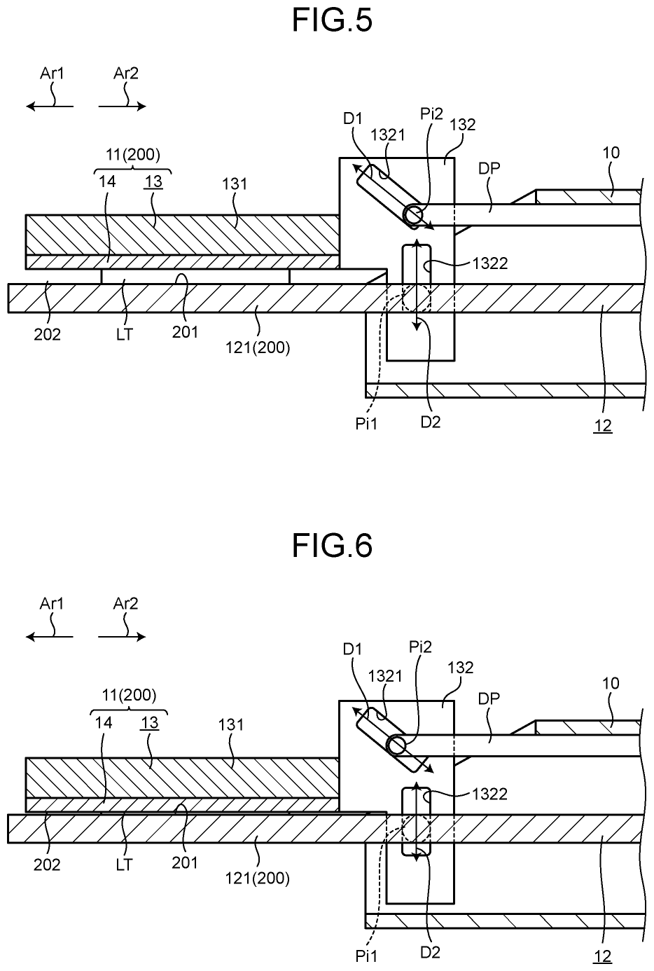

Furthermore, when the driver DP further moves to the distal end side Ar1 in response to the opening/closing operation from the state in FIG. 4 illustrating a point of time at which the jaw 11 and the treatment portion 121 start gripping the treatment target LT, and the jaw 11 is further closed toward the treatment portion 121, the pair of bearing portions 132 that are the end portions on the proximal end side Ar2 of the jaw 11 move in a direction to move away from the treatment portion 121 along the second direction D2 while being guided to the second elongated hole 1322 as illustrated in FIG. 5. At this time, as illustrated in FIG. 5, the position of the driving shaft Pi2 in the first elongated hole 1321 is also changed. As a result, the first and second gripping surfaces 201 and 202 become parallel to each other.

In a state where the first and second gripping surfaces 201 and 202 are parallel, the first direction D1 is an inclined direction that is a direction to move away from the treatment portion 121 toward the distal end side Ar1 as illustrated in FIG. 5. On the other hand, the second direction D2 is a direction parallel to a direction in which the first and second gripping surfaces 201 and 202 face each other (a normal direction of first and second gripping surfaces 201 and 202).

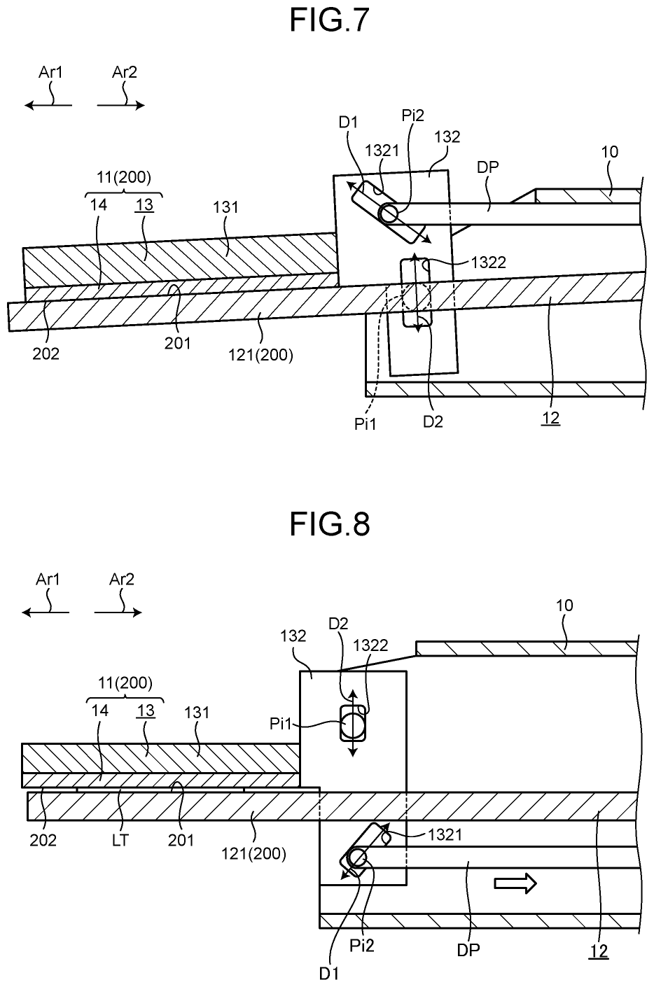

When the driver DP further moves to the distal end side Ar1 in response to the opening/closing operation from the state in FIG. 5 illustrating the state where the first and second gripping surfaces 201 and 202 are parallel, and the jaw 11 further is closed toward the treatment portion 121, the driving shaft Pi2 moves to the distal end side Ar1 along the first direction D1 while being guided to the first elongated hole 1321 as illustrated in FIGS. 6 and 7. Furthermore, the pair of bearing portions 132 move in a direction to approach the treatment portion 121 along the second direction D2 while being guided to the second elongated hole 1322. That is, as can be seen from FIGS. 5 to 7, the first and second gripping surfaces 201 and 202 increase the gripping force relative to the treatment target LT while maintaining the state where the first and second gripping surfaces 201 and 202 are parallel to each other. At this time, the vibration transmitter 12 is warped in proportion to the gripping force as illustrated in FIG. 7.

The above-described present embodiment provides the following effects.

According to the treatment tool 2 according to the present embodiment, as the driver DP moves forward and backward, the driving shaft Pi2 moves along the first direction D1 in the first elongated hole 1321 while being guided to the first elongated hole 1321. Furthermore, as the driving shaft Pi2 moves along the first direction D1 in the first elongated hole 1321, the jaw 11 moves along the second direction D2 relative to the pivoting shaft Pi1 while being guided to the second elongated hole 1322. More specifically, when the jaw 11 is further closed toward the treatment portion 121 from the point of time at which the jaw 11 and the treatment portion 121 have started gripping the treatment target LT, the pair of bearing portions 132 move in the direction to move away from the treatment portion 121 along the second direction D2 relative to the pivoting shaft Pi1 while being guided to the second elongated hole 1322. Furthermore, when the jaw 11 is closed toward the treatment portion 121 from the state where the first and second gripping surfaces 201 and 202 are parallel, the pair of bearing portions 132 move in the direction to approach the treatment portion 121 along the second direction D2 relative to the pivoting shaft Pi1 while being guided to the second elongated hole 1322.

Therefore, even when the treatment target LT is thin and soft living tissues, the gripping pressure on the distal end side Ar1 does not become higher than that of other sites, and even when the treatment target LT is thick and hard living tissues, the gripping pressure on the proximal end side Ar2 does not become higher than that of other sites. Therefore, the treatment tool 2 according to the present embodiment can reduce variations in a gripping pressure distribution when gripping the treatment target LT.

In particular, the first direction D1 that is the extension direction of the first elongated hole 1321 is the inclined direction that is the direction to move away from the treatment portion 121 toward the distal end side Ar1 in the state where the first and second gripping surfaces 201 and 202 are parallel.

Consequently, it is possible to apply a force from the driving shaft Pi2 to the jaw 11 in the direction to approach the treatment portion 121, and it is possible to uniformly increase the gripping force relative to the treatment target LT while maintaining the state where the first and second gripping surfaces 201 and 202 are parallel to each other.

### Other Embodiments

Although the mode for carrying out the disclosure has been described above, the disclosure should not be limited only to the above-described embodiment.

Although ultrasound energy and high frequency energy are adopted as treatment energy applied to the treatment target LT by the treatment tool 2 in the above-described embodiment, the disclosure is not limited thereto, and only the ultrasound energy may be used.

Although the pivoting shaft Pi1 is formed as the pin in the above-described embodiment, the disclosure is not limited thereto, and the pivoting shaft Pi1 may be formed as a protrusion that protrudes from the sheath 10. Similarly, the driving shaft Pi2 may also be formed as a protrusion that protrudes from the driver DP.

### Modified Example 1

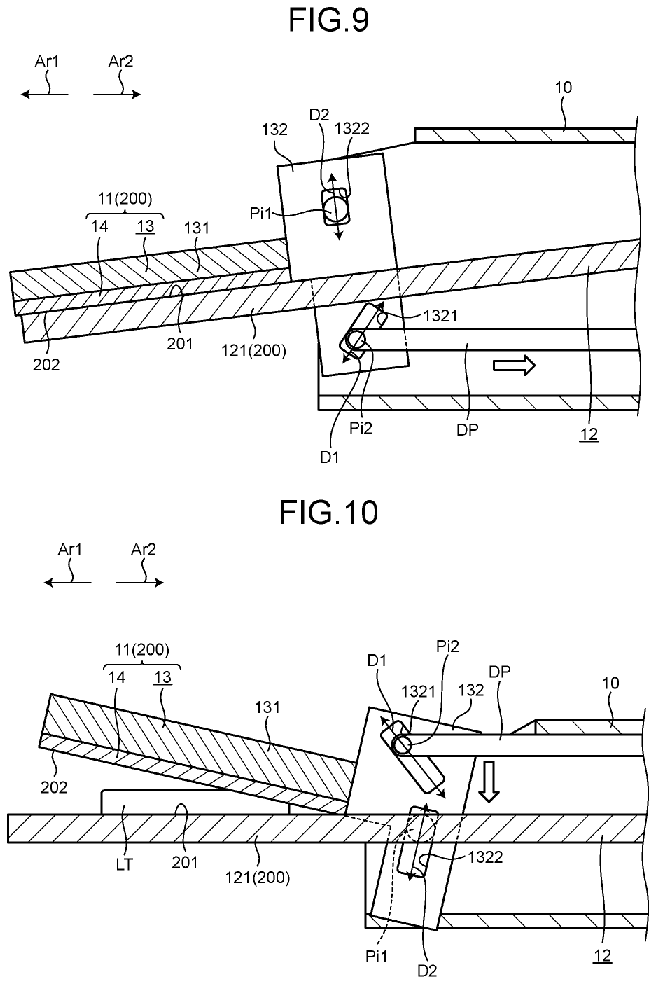

FIGS. 8 and 9 are views illustrating modified example 1 of the embodiment. More specifically, FIG. 8 is the view corresponding to FIG. 6. FIG. 9 is the view corresponding to FIG. 7.

Although the treatment tool 2 is formed as a so-called push-close type in the above-described embodiment, the disclosure is not limited thereto, and the treatment tool 2 may be formed as a so-called pull-close type as in this modified example 1 illustrated in FIGS. 8 and 9.

More specifically, in this modified example 1, as illustrated in FIGS. 8 and 9, the jaw 11 pivots about the pivoting shaft Pi1 in the direction to approach the treatment portion 121 in conjunction with movement of the driver DP to the proximal end side Ar2. That is, the jaw 11 is closed toward the treatment portion 121. Furthermore, the jaw 11 pivots about the pivoting shaft Pi1 in the direction to move away from the treatment portion 121 in conjunction with movement of the driver DP to the distal end side Art. That is, the jaw 11 is opened from the treatment portion 121.

Note that, in order to configure the treatment tool as the so-called pull-close type, the positional relationship between the pivoting shaft Pi1 and the driving shaft Pi2 according to this modified example 1 and the positional relationship between the first and second elongated holes 1321 and 1322 according to this modified example 1 are set reversely from those of the above-described embodiment as illustrated in FIGS. 8 and 9.

Even in the case where the treatment tool is formed as the so-called pull-close type in this above-described modified example 1, it is possible to obtain the same effect as those of the above-described embodiment.

### Modified Example 2

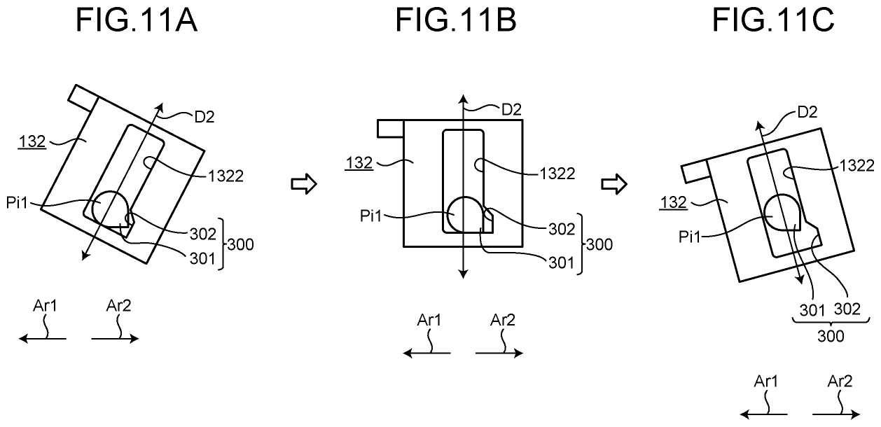

FIG. 10 and FIGS. 11A to 11C are views for describing modified example 2 of the embodiment. More specifically, FIG. 10 is a view corresponding to FIG. 3, and is the view for describing a problem that may occur in the above-described embodiment. FIGS. 11A to 11C are the views illustrating the enlarged pivoting shaft Pi1 and second elongated hole 1322 according to this modified example 2. Note that FIG. 11A is a view illustrating the pivoting shaft Pi1 and the second elongated hole 1322 in the state in FIG. 3 illustrating the state where the operator such as the surgeon does not perform the opening/closing operation of the operation handle 7. FIG. 11B is a view illustrating the pivoting shaft Pi1 and the second elongated hole 1322 in the state in FIG. 5 illustrating the state where the first and second gripping surfaces 201 and 202 are parallel. FIG. 11C is a view illustrating the pivoting shaft Pi1 and the second elongated hole 1322 in the states in FIGS. 6 and 7 illustrating the states where the jaw 11 is further closed toward the treatment portion 121 from the state in FIG. 5.

Meanwhile, in the above-described embodiment, there is, for example, a case where the jaw 11 is in the state illustrated in FIG. 10 in a state where the operator such as the surgeon does not perform the opening/closing operation on the operation handle 7. More specifically, the state illustrated in FIG. 10 is a state where the pair of bearing portions 132 have moved in the direction to approach the treatment portion 121 along the second direction D2 while being guided to the second elongated hole 1322. Furthermore, when the jaw 11 is closed toward the treatment portion 121 from the state illustrated in FIG. 10, the second gripping surface 202 starts coming into contact with the first gripping surface 201 from the proximal end side Ar2. Therefore, there is a problem (hereinafter, referred to as a sinking problem) that the gripping pressure distribution at a time when the treatment target LT is gripped becomes an unintended gripping pressure distribution.

Therefore, in this modified example 2, in order to solve the above-described sinking problem, an engagement structure 300 is provided to the pivoting shaft Pi1 and the second elongated hole 1322 as illustrated in FIGS. 11A to 11C.

As illustrated in FIGS. 11A to 11C, the engagement structure 300 includes a protrusion portion 301 and a recess portion 302.

The protrusion portion 301 is a protrusion that is provided on part of the outer peripheral surface of the pivoting shaft Pi1.

The recess portion 302 is a recess portion that is provided on part of the inner surface of the second elongated hole 1322, and into which the protrusion portion 301 can be inserted.

Furthermore, in a first operation state, the engagement structure 300 forbids the pair of bearing portions 132 from moving in the direction to approach the treatment portion 121 along the second direction D2 while being guided to the second elongated hole 1322.

More specifically, the first operation state is an operation state where from the jaw 11 starts closing toward the treatment portion 121 until the first and second gripping surfaces 201 and 202 enter a parallel state. That is, the first operation state is the operation state from the state illustrated in FIG. 11A to the state illustrated in FIG. 11B. In the first operation state, the protrusion portion 301 enters the recess portion 302 as illustrated in FIG. 11A. Therefore, the pair of bearing portions 132 cannot move in the direction (the lower side in FIGS. 11A to 11C) to approach the treatment portion 121 along the second direction D2 while being guided to the second elongated hole 1322.

Furthermore, in a second operation state, the engagement structure 300 permits the pair of bearing portions 132 to move in the direction to approach the treatment portion 121 along the second direction D2 while being guided to the second elongated hole 1322.

More specifically, the second operation state is an operation state where the jaw 11 is closed toward the treatment portion 121 from a state where the first and second gripping surfaces 201 and 202 are parallel. That is, the second operation state is the operation state from the state illustrated in FIG. 11B to the state illustrated in FIG. 11C. In the second operation state, as illustrated in FIGS. 11B and 11C, the protrusion portion 301 is located outside the recess portion 302 by rotation of the jaw 11 about the pivoting shaft Pi1. Consequently, the pair of bearing portions 132 can move in the direction (the lower side in FIGS. 11A to 11C) to approach the treatment portion 121 along the second direction D2 while being guided to the second elongated hole 1322.

In a case where the configuration including the engagement structure 300 is adopted as in this above-described modified example 2, it is possible to provide an effect that it is possible to solve the above-described sinking problem in addition to the effect similar to the above-described embodiment.

### Modified Example 3

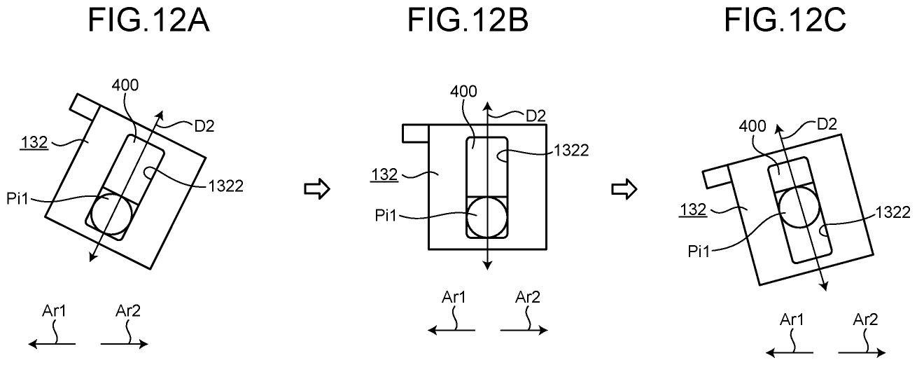

FIGS. 12A to 12C are views for describing modified example 3 of the embodiment. More specifically, FIGS. 12A to 12C are views illustrating the enlarged pivoting shaft Pi1 and second elongated hole 1322 according to this modified example 3. Note that FIG. 12A is a view illustrating the pivoting shaft Pi1 and the second elongated hole 1322 in the state in FIG. 3 illustrating the state where the operator such as the surgeon does not perform the opening/closing operation of the operation handle 7. FIG. 12B is a view illustrating the pivoting shaft Pi1 and the second elongated hole 1322 in the state in FIG. 5 where the first and second gripping surfaces 201 and 202 are parallel. FIG. 12C is a view illustrating the pivoting shaft Pi1 and the second elongated hole 1322 in the states in FIGS. 6 and 7 where the jaw 11 is further closed toward the treatment portion 121 from the state in FIG. 5.

In this modified example 3, in order to solve the above-described sinking problem, an elastic material 400 is adopted instead of the engagement structure 300 according to above-described modified example 2.

As illustrated in FIGS. 12A to 12C, the elastic material 400 is disposed on the upper side of the pivoting shaft Pi1 in the second elongated hole 1322 in FIGS. 12A to 12C.

Furthermore, in the first operation state, the elastic material 400 forbids the pair of bearing portions 132 from moving in the direction to approach the treatment portion 121 along the second direction D2 while being guided to the second elongated hole 1322.

More specifically, the first operation state is an operation state where from the jaw 11 starts closing toward the treatment portion 121 until the first and second gripping surfaces 201 and 202 enter a parallel state. That is, the first operation state is an operation state from the state illustrated in FIG. 12A to the state illustrated in FIG. 12B. In the first operation state, the pair of bearing portions 132 cannot move in the direction (the lower side in FIGS. 11A to 11C) to approach the treatment portion 121 along the second direction D2 while being guided to the second elongated hole 1322 by the elastic force of the elastic material 400.

Furthermore, in the second operation state, the elastic material 400 permits the pair of bearing portions 132 to move in the direction to approach the treatment portion 121 along the second direction D2 while being guided to the second elongated hole 1322.

More specifically, the second operation state is an operation state where the jaw 11 is closed toward the treatment portion 121 from a state where the first and second gripping surfaces 201 and 202 are parallel. That is, the second operation state is an operation state from the state illustrated in FIG. 12B to the state illustrated in FIG. 12C. When a downward force applied from the driving shaft Pi2 to the inner surface of the first elongated hole 1321 in FIGS. 12A to 12C exceeds the elastic force of the elastic material 400, the elastic material 400 is compressed in the second operation state, so that the pair of bearing portions 132 can move in the direction (the lower side in FIGS. 12A to 12C) to approach the treatment portion 121 along the second direction D2 while being guided to the second elongated hole 1322.

In the case where the configuration including the elastic material 400 is employed as in this above-described modified example 3, it is possible to provide an effect that it is possible to solve the above-described sinking problem in addition to the effect similar to the above-described embodiment.

### Modified Example 4

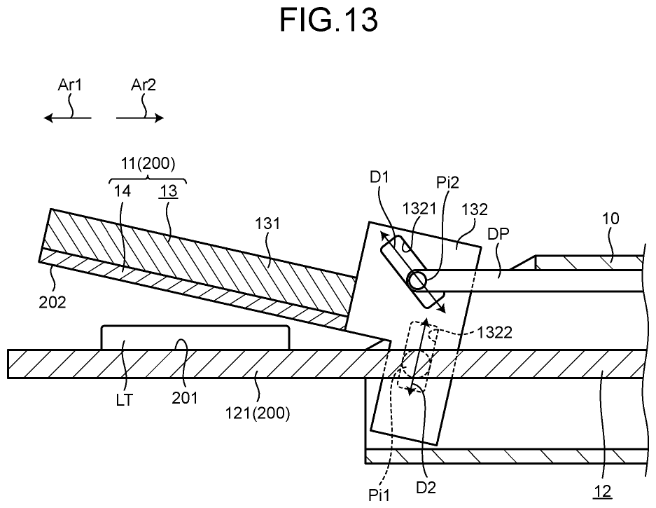

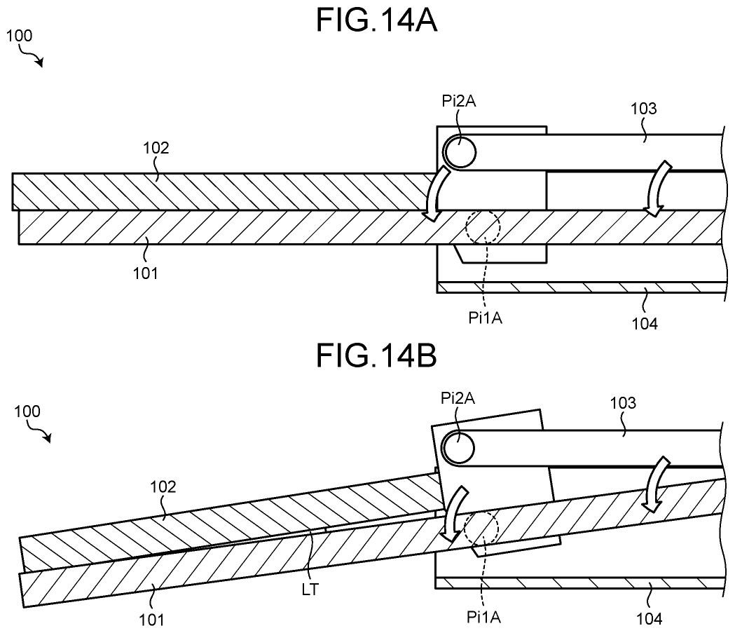

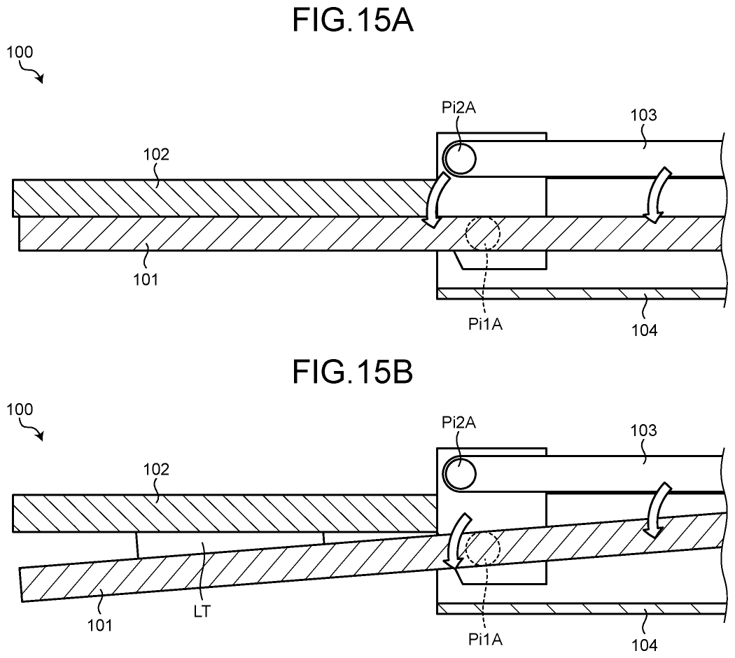

FIG. 13 is a view for describing modified example 4 of the embodiment. More specifically, FIG. 13 is the view corresponding to FIG. 3.

Although the pivoting shaft Pi1 is fixed to the sheath 10, and the second elongated hole 1322 is provided to the bearing portion 132 in the above-described embodiment, the disclosure is not limited thereto. As illustrated in, for example, FIG. 13, the pivoting shaft Pi1 may be provided to the bearing portion 132, and the second elongated hole 1322 may be provided to the sheath 10 contrary to the above-described embodiment.

Even in a case where the configuration of this above-described modified example 4 is employed, it is possible to provide the same effect as those of the above-described embodiment.

Similarly, contrary to the above-described embodiment, the driving shaft Pi2 may be provided to the bearing portion 132, and the first elongated hole 1321 may be provided to the driver DP.

The treatment tool according to the disclosure can reduce variations in a gripping pressure distribution when living tissues are gripped.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

In this document the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the term “and/or” is used to refer to a nonexclusive or, such that “A and/or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the embodiments should be determined with reference to the appended claims, along with the full scope of equivalents to which such claims are entitled.

What is claimed is:

1. A treatment tool comprising:
a first gripper;
a second gripper configured to pivot relative to the first gripper to grip a living tissue between the second gripper and the first gripper;
a support provided with a pivoting shaft configured to engage with the second gripper and serve as a pivoting center of the second gripper; and
a driver provided with a driving shaft configured to engage with the second gripper, the driver configured to move forward and backward to pivot the second gripper, wherein the second gripper includes:
a first elongated hole being an elongated hole extending along a first direction, in which the driving shaft is disposed, the first elongated hole engaging with the driving shaft; and
a second elongated hole being an elongated hole extending along a second direction, in which the pivoting shaft is disposed, the second elongated hole engaging the pivoting shaft, wherein as the driver moves forward and backward, the driving shaft is configured to move along the first direction in the first elongated hole while being guided to the first elongated hole, and wherein as the driving shaft moves along the first direction in the first elongated hole, the second gripper is configured to move along the second direction relative to the pivoting shaft while being guided to the second elongated hole.

2. The treatment tool according to claim 1, wherein, when the driver moves in a direction to approach the second gripper, the second gripper is configured to pivot in a direction to approach the first gripper.

3. The treatment tool according to claim 1, wherein the first gripper includes a first gripping surface, wherein the second gripper includes a second gripping surface configured to grip the living tissue between the second gripping surface and the first gripping surface.

4. The treatment tool according to claim 3, wherein when the second gripper pivots in a direction to approach the first gripper from a state where the first gripping surface and the second gripping surface are parallel, the driving shaft is configured to move along the first direction in the first elongated hole while being guided to the first elongated hole.

5. The treatment tool according to claim 3, wherein the first direction is an inclined direction being a direction to move away from the first gripper toward a distal end of the second gripper in a state where the first gripping surface and the second gripping surface are parallel.

6. The treatment tool according to claim 1, the first elongated hole is inclined relative to the second elongated hole.

7. The treatment tool according to claim 1, wherein the first gripper is a vibration transmitter configured to transmit ultrasound vibration.

8. The treatment tool according to claim 7, wherein the first gripper and the second gripper are electrodes configured to cause a high frequency current to flow to the living tissue.

9. The treatment tool according to claim 1, wherein the support has a tubular shape, and wherein the driver is inserted into the support.

10. The treatment tool according to claim 3, wherein an outer surface of the pivoting shaft and an inner surface of the second elongated hole are provided with an engagement structure configured to forbid movement of the second gripper along the second direction relative to the pivoting shaft in a first operation state and permit movement of the second gripper along the second direction relative to the pivoting shaft in a second operation state, wherein the first operation state is an operation state where from the second gripper pivots in a direction to approach the first gripper until the first gripping surface and the second gripping surface enter a parallel state, and wherein the second operation state is an operation state where the second gripper pivots in the direction to approach the first gripper from a state where the first gripping surface and the second gripping surface are parallel.

11. The treatment tool according to claim 3, wherein an inside of the second elongated hole is provided with an elastic material configured to forbid movement of the second gripper along the second direction relative to the pivoting shaft in a first operation state and permit movement of the second gripper along the second direction relative to the pivoting shaft in a second operation state, wherein the first operation state is an operation state where from the second gripper pivots in a direction to approach the first gripper until the first gripping surface and the second gripping surface enter a parallel state, and wherein the second operation state is an operation state where the second gripper pivots in the direction to approach the first gripper from a state where the first gripping surface and the second gripping surface are parallel.

12. The treatment tool according to claim 1, wherein, when the driver moves in a direction to move away from the second gripper, the second gripper is configured to pivot in a direction to approach the first gripper.

13. The treatment tool according to claim 1, wherein the support has a tubular shape, and wherein the first gripper is inserted into the support and disposed protruding from a distal end of the support.

14. The treatment tool according to claim 13, wherein the driver is inserted into the support and has a tubular shape, and wherein the first gripper is inserted into the driver.

15. The treatment tool according to claim 1, wherein the second gripper includes:
a gripper main body provided with the first elongated hole and the second elongated hole; and
a pad provided to the gripper main body, the pad configured to be in contact with the first gripper when the second gripper pivots in a direction to approach the first gripper.

16. The treatment tool according to claim 1, further comprising:
a fixed handle gripped by a user; and
an operation handle movable relative to the fixed handle, and the operation handle accepts an operation of the user, wherein the driver configured to move forward and backward in response to an operation of the operation handle by the user.

17. The treatment tool according to claim 1, wherein the first elongated hole and the second elongated hole are provided at an end portion on a proximal end side of the second gripper, and wherein as the second gripper pivots in a direction to approach the first gripper from a point of time when the first gripper and the second gripper grip the living tissue, the end portion on the proximal end side of the second gripper is configured to move in a direction to move away from the first gripper along the second direction relative to the pivoting shaft while being guided to the second elongated hole.

18. The treatment tool according to claim 1, wherein the first elongated hole and the second elongated hole are provided at an end portion on a proximal end side of the second gripper, wherein the first gripper includes a first gripping surface, wherein the second gripper includes a second gripping surface configured to grip the living tissue between the second gripping surface and the first gripping surface, and wherein when the second gripper pivots in the direction to approach the first gripper from a state where the first gripping surface and the second gripping surface are parallel, the end portion on the proximal end side of the second gripper is configured to move in the direction to approach the first gripper along the second direction relative to the pivoting shaft while being guided to the second elongated hole.

19. A treatment tool comprising:
a first gripper;
a second gripper including a pivoting shaft (Pi1A), the second gripper being configured to pivot about the pivoting shaft relative to the first gripper to grip a living tissue between the second gripper and the first gripper;
a support configured to engage with the pivoting shaft; and
a driver including a driving shaft configured to engage with the second gripper, the driver configured to move forward and backward to pivot the second gripper, wherein the second gripper includes a first elongated hole being an elongated hole extending along a first direction, in which the driving shaft is disposed, the first elongated hole engaging the driving shaft, wherein the support includes a second elongated hole being an elongated hole extending along a second direction, in which the pivoting shaft is disposed, the second elongated hole engaging with the pivoting shaft, wherein as the driver moves forward and backward, the driving shaft is configured to move along the first direction in the first elongated hole while being guided to the first elongated hole, and wherein as the driving shaft moves along the first direction in the first elongated hole, the second gripper is configured to move along the second direction while the pivoting shaft is guided to the second elongated hole.

20. The treatment tool according to claim 19, the first elongated hole is inclined relative to the second elongated hole.
